(19) United States
(12) Patent Application Publication
(10) Pub. No.: US 2020/0010395 A1
(43) Pub. Date: Jan. 9, 2020

(54) METHOD FOR PREPARING A ß-HYDROXYKETONE

(71) Applicant: BASF SE, Ludwigshafen am Rhein (DE)

(72) Inventors: Dagmar Pascale KUNSMANN-KEITEL, Ludwigshafen am Rhein (DE); Georg Heinrich GROSCH, Ludwigshafen am Rhein (DE); Jan-Dirk ARNDT, Ludwigshafen am Rhein (DE); Gauthier Luc Maruice AVERLANT, Darmstadt (DE); Wolf-Steffen WEISSKER, Ludwigshafen am Rhein (DE); Marcus Georg SCHREMS, Ludwigshafen am Rhein (DE); Dan-Tam Daniel TANG, Ludwigshafen am Rhein (DE)

(21) Appl. No.: 16/460,402

(22) Filed: Jul. 2, 2019

(30) Foreign Application Priority Data
Jul. 6, 2018 (EP) 18182097.8

(51) Int. Cl. C07C 45/64; C07C 49/17; C07C 45/83

(52) U.S. Cl. CPC C07C 45/64; C07C 45/83; C07C 49/17

(57) ABSTRACT

Method for preparing a β-hydroxyketone having 4 to 8 carbon atoms by reacting formaldehyde with a branched or unbranched dialkyl ketone having 3 to 7 carbon atoms in the liquid phase in a reactor in the presence of a basic component at a temperature of 50 to 150° C. and a pressure of 0.2 to 10 MPa abs, in which (a) a trialkylamine having 1 to 4 carbon atoms per alkyl group is used as basic component and the reaction (b) is carried out in the presence of 1 to 25% by weight water, based on the liquid phase, and (c) at a molar ratio of trialkylamine to formaldehyde in the liquid phase of from 1 to 5.

### CROSS-REFERENCE TO RELATED APPLICATIONS

This application claims priority to European Application No. 18182097.8, filed Jul. 6, 2018, which is incorporated herein by reference in its entirety.

The present invention relates to a method for preparing a β-hydroxyketone having 4 to 8 carbon atoms by reacting formaldehyde (II) with a branched or unbranched dialkyl ketone having 3 to 7 carbon atoms in the liquid phase in a reactor in the presence of a basic component at a temperature of 50 to 150° C. and a pressure of 0.2 to 10 MPa abs.

β-Hydroxyketones, such as 1-hydroxy-2-methyl-3-pentanone for example, can be used as solvents, owing to their polar property, for example for natural and synthetic resin varnishes. Due to their hydroxyl and keto group, β-hydroxyketones are, however, also of interest as synthetic units for the preparation of active ingredients. In addition, they can be converted into the corresponding enones by elimination of water, which are also interesting synthetic units.

From a reaction engineering perspective, the aldol addition of formaldehyde to the appropriate dialkyl ketones provides the industrial synthesis of β-hydroxyketones. For instance, 1-hydroxy-2-methyl-3-pentanone can be obtained by aldol addition of formaldehyde to diethyl ketone.

Aldol additions are well-known to those skilled in the art. In aldol addition, aldehydes and ketones react to give β-hydroxyaldehydes or β-hydroxyketones. They can be be carried out base-catalyzed or acid-catalyzed. In the case of a base-catalysed aldol addition, an enolate anion is initially formed by base-catalyzed H⁺ elimination at the α-position, which in the form of its nucleophilic methylene component then adds to the second carbonyl component with addition of H⁺ and recovery of the basic component.

U.S. Pat. No. 3,662,001 describes the synthesis of 3-keto-1-butanol by aldol addition of formaldehyde to acetone and teaches the use of basic catalysts as advantageous. In this case, trialkylamines, inter alia, are also mentioned as suitable.

PL 180,623 B1 teaches the implementation of the aldol addition of formaldehyde to low-molecular weight ketones, such as acetone for example, in the presence of a tertiary amine such as triethylamine, to give the corresponding β-hydroxymethylketone and derivatives thereof. In terms of ratio, the document teaches the use of 0.5 to 15 g of tertiary amine with 1 to 13 mol of formaldehyde, which corresponds to a molar ratio of triethylamine to formaldehyde of 0.00038 to 0.148. In the case of trimethylamine as simplest tertiary amine, the ranges stated correspond to a molar ratio of trimethylamine to formaldehyde of 0.00065 to 0.254.

DE 1,952,738 describes the preparation of trimethylolpropane by two-fold aldol addition of formaldehyde to n-butyraldehyde in the presence of a low-molecular weight organic tertiary amine and subsequent Cannizzaro reaction for the reduction of the intermediate 2,2-dimethylolbutyraldehyde formed. In the aldol addition in example 1, a molar ratio of trimethylamine to formaldehyde of 0.236 was used and in example 2 a molar ratio of triethylamine to formaldehyde of 0.259 was used.

US 2008/0,004,475, US 2013/0,053,534 and U.S. Pat. No. 8,394,998 disclose the two-stage synthesis of neopentyl glycol by aldol addition of formaldehyde to isobutyraldehyde in the presence of a trialkylamine with formation of hydroxypivaldehyde and subsequent catalytic hydrogenation with hydrogen to give neopentyl glycol. US 2008/0,004,475 and US 2013/0,053,534 teach for the aldol addition in this case a molar ratio of isobutyraldehyde to formaldehyde from 1:2 to 1:5 and a molar ratio of trialkylamine to isobutyraldehyde of 0.001 to 0.2, which corresponds to a molar ratio of trialkylamine to formaldehyde of 0.0002 to 0.1. In the examples, a molar ratio of trialkylamine to formaldehyde of 0.060 was used in each case. For the aldol addition, U.S. Pat. No. 8,394,998 teaches the use of molar amounts of isobutyraldehyde and formaldehyde and also a molar ratio of trialkylamine to isobutyraldehyde of 0.01 to 0.2, which corresponds to a molar ratio of trialkylamine to formaldehyde of 0.01 to 0.2.

U.S. Pat. No. 3,077,500 also teaches the use of a trialkylamine as catalyst in base-catalyzed adol addition. Example X describes here the reaction of methyl ethyl ketone with formaldehyde to give 4-hydroxy-3-methyl-2-butanone in the presence of triethylamine in a continuously operated reaction system at a molar ratio of triethylamine to formaldehyde in the reactor inlet of 0.396.

The documents cited teach the use of catalytic amounts of trialkylamine in the base-catalyzed aldol addition of formaldehyde to aldehydes and ketones in the synthesis of β-hydroxyaldehydes and β-hydroxyketones as intermediates. An essential element of the aldol addition described is the use of only a relatively low amount of trialkylamine of less than 0.4 mol per mole of formaldehyde, since this low amount already enables a sufficiently rapid reaction rate. This appears advantageous for several reasons. For instance, if less trialkylamine is initially provided, less trialkylamine is to be removed after reaction is complete. In addition, this mode of operation also enables a more highly concentrated reaction solution which in turn permits a smaller reaction apparatus.

Furthermore, many aldehydes and ketones are readily accessible as synthetic units and aldol addition can be carried out relatively easily from a reaction engineering perspective and, most importantly, low-boiling trialkylamines can be readily removed from the reaction mixture by distillation.

Also for the synthesis of 1-hydroxy-2-methyl-3-pentanone (Ia), the base-catalyzed aldol addition of formaldehyde (II) to diethyl ketone (IIIa) appears advisable from a reaction engineering perspective, especially as the basic catalyst can be selected by using a tertiary amine such that the latter can be easily removed as low boiler by subsequent distillation and can be recycled. The reaction equation using a trialkylamine as catalyst is therefore

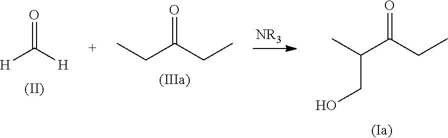

wherein NR₃ is a trialkylamine with the same or different alkyl radicals having 1 to 4 carbon atoms in each case. Typically, the reaction would be operated at elevated temperature (above room temperature) and elevated temperature (above atmospheric pressure).

A typical secondary reaction in aldol addition of formaldehyde to ketones is the multiple addition of formaldehyde.

In accordance with the invention, it was recognized that the stated multiple addition of formaldehyde with all its negative effects also occurs in the case of aldol addition of formaldehyde to diethyl ketone to give 1-hydroxy-2-methyl-3-pentanone. For instance, particularly the dihydroxy compounds 1,5-dihydroxy-2,4-dimethyl-3-pentanone (IVa) and 1-hydroxy-2-hydroxymethyl-2-methyl-3-pentanone (Va) and also the trihydroxy compound 1,5-dihydroxy-2-hydroxymethyl-2,4-dimethyl-3-pentanone (VIa) are thereby formed.

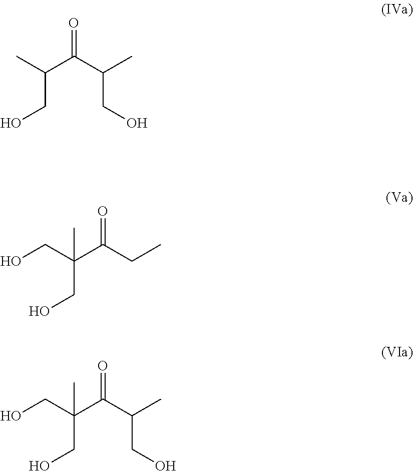

β-Hydroxyketones, such as the specific compound (Ia) for example, but also the di- and trihydroxy compounds formed by multiple addition of formaldehyde to dialkyl ketone, such as the specific compounds (IVa), (Va) and (VIa) for example, generally tend to eliminate water to form an α,β-unsaturated ketone (enone) in the presence of acids or bases in the case of supply of heat.

For instance, the textbook Organic Chemistry: Structure, Mechanism, and Synthesis, Robert J. Ouellette and J. David Rawn, Elsevier 2014, ISBN 978-0-12-800780-8 in chapter 22.6 describes the base-catalyzed aldol condensation of aldehydes with elimination of water to give the corresponding enals. In a first step, the addition product is initially formed, which then dehydrates at elevated temperature to the enal. In addition to the dehydration in the presence of a catalytic amount of a base, the dehydration can also be catalyzed by strong acids.

The textbook Organic Chemistry, John McMurry, 5th edition, Brooks/Cole 2000, ISBN 0-534-37366-6 in chapter 23.4 describes the dehydration of β-hydroxyaldehydes and β-hydroxyketones to the corresponding enals and enones. The dehydration can be base-catalyzed with elimination of an acidic α-hydrogen and subsequent elimination of an OH⁻ group, and acid-catalyzed with protonation of the ═O group and also the OH group, elimination of an α-hydrogen and subsequent elimination of an H₃O⁺ group. The document teaches that somewhat more forceful conditions are often required for dehydration, such as a somewhat elevated temperature, than in the preceding aldol addition.

Both textbooks therefore teach that, in addition to catalytically active amounts of a base or an acid, the formation of enals and enones from β-hydroxyaldehydes and β-hydroxyketones particularly require elevated temperatures. For instance, the enone 2-methyl-1-penten-3-one (VIIa) can be formed by dehydration of 1-hydroxy-2-methyl-3-pentanone (Ia) and the enone 2,4-dimethyl-5-hydroxy-1-penten-3-one (VIIIa) can be formed by dehydration of 1,5-dihydroxy-2,4-dimethyl-3-pentanone (IVa).

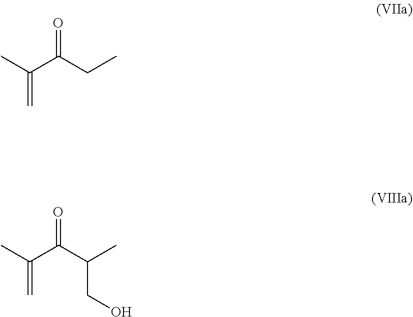

Since the enones formed are unsaturated reactive compounds, these tend to further reaction, such as to dimerization, oligomerization or even polymerization for example.

The secondary reactions mentioned above are disadvantageous for many reasons and therefore undesirable.
1. Firstly, the secondary reactions are practically irreversible and reduce the yield of target product, that is to say the desired β-hydroxyketone.
2. Secondly, the secondary reactions increase the amounts used of the dialkyl ketone and formaldehyde reactants since these are bound practically irreversibly in the by-products.
3. Thirdly, the loss of formaldehyde is further aggravated by the fact that even two or three moles of formaldehyde per mole of dialkyl ketone are bound in the form of by-products by the formation of di- and trihydroxy compounds as by-products.
4. Fourthly, the by-products increase the complexity of the workup of the reaction mixture since these are to be removed from the β-hydroxyketone target product.
5. Fifthly, the risk exists that undesired deposits form on the apparatus due to oligomeric or polymeric by-products and these have to be cleaned from time to time.
6. Sixthly, due to the diversity of the various by-products, there may also be by-products which can only be removed with great difficulty from the β-hydroxyketone target product. For instance, it was recognized very specifically in accordance with the invention that 2,4-dimethyl-5-hydroxy-1-penten-3-one (VIIIa) having a boiling point of 209° C. at 0.1 MPa abs (or of ca. 233° C. at 0.2 MPa abs) can only be separated with great difficulty by distillation due to its boiling point from 1-hydroxy-2-methyl-3-pentanone (I), which has a boiling point of 202° C. at 0.1 MPa abs (or of ca. 231° C. at 0.2 MPa abs).

It is therefore desirable in the preparation of β-hydroxyketones to significantly reduce the formation of these by-products mentioned.

It was therefore the object of the present invention to find a method for preparing β-hydroxyketones, which is based on readily accessible feedstocks, is simple and safe to carry out, enables the highest possible selectivity, yield and purity of product of value, and in particular largely avoids or at least significantly reduces the aforementioned disadvantages due to formation of undesired by-products with reduction of the yield, increase in the amounts used and increase in the complexity for separation and disposal. In addition, the method should also have a high space-time yield in order to use the necessary apparatus most efficiently.

Surprisingly, a method has been found for preparing a β-hydroxyketone (I) having 4 to 8 carbon atoms by reacting formaldehyde (II) with a branched or unbranched dialkyl ketone (III) having 3 to 7 carbon atoms in the liquid phase in a reactor in the presence of a basic component at a temperature of 50 to 150° C. and a pressure of 0.2 to 10 MPa abs, in which (a) a trialkylamine having 1 to 4 carbon atoms per alkyl group is used as basic component and the reaction (b) is carried out in the presence of 1 to 25% by weight water, based on the liquid phase, and (c) at a molar ratio of trialkylamine to formaldehyde (II) in the liquid phase of 1 to 5.

### A BRIEF DESCRIPTION OF THE FIGURE

FIG. 1 illustrates an exemplary representation of a continuously operated plant according to the invention.

In the method according to the invention, formaldehyde (II) is reacted with a branched or unbranched dialkyl ketone (III) having 3 to 7 carbon atoms to give the corresponding β-hydroxyketone (I) under the conditions stated above.

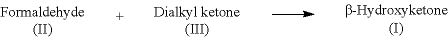

As explained by way of introduction in the example of 1-hydroxy-2-methyl-3-pentanone (Ia) as β-hydroxyketone (I), the aldol addition does not necessarily stop at the β-hydroxyketone (I), but reacts to some extent by further additions of formaldehyde (II) to give the corresponding di- and trihydroxy compounds. The β-hydroxyketo group is common to the β-hydroxyketone (I) and the di- and trihydroxy compounds. These can lead by dehydration to the corresponding enone, which again as reactive molecule has a tendency to dimerization and further secondary reactions. Accordingly, there is thus diversity in the typical by-product spectrum in the aldol addition specified.

The method according to the invention is characterized in particular by the use of a relatively high molar amount of trialkylamine with respect to the molar amount of formaldehyde (II).

Since the trialkylamine is only a catalyst for promoting the reaction specified and is therefore also not consumed, it would actually be obvious to keep the amount of trialkylamine as low as possible. This is also supported by the fact that even relatively low amounts of trialkylamine already enable a correspondingly high conversion. Also with regard to the process economy, a low amount of trialkylamine would be used anyway.

Contrary to any expectation however, it has been shown that, surprisingly, by using such a high molar ratio of trialkylamine to formaldehyde (II) in the liquid phase of from 1 to 5 in the reaction of formaldehyde (II) with dialkyl ketone (III), significantly fewer undesirable by-products are formed than when using a substantially lower molar ratio of less than 0.4 mol of trialkylamine per mole of formaldehyde in accordance with the prior art.

In the present invention, the molar ratio according to the invention of trialkylamine to formaldehyde (II) in the liquid phase is from 1 to 5, particularly preferably ≥1.1 and especially preferably ≥1.2 and preferably ≤3 and particularly preferably ≤2. Therefore, the molar amount of trialkylamine catalyst according to the invention is sometimes even significantly above the molar amount of the reactant formaldehyde (II). The molar ratio of trialkylamine to formaldehyde (II) in the liquid phase is preferably from 1 to 3.

In connection with the investigations of the molar ratio of trialkylamine with respect to the molar amount of formaldehyde (II), it has also been shown that, surprisingly, a high molar ratio according to the invention promotes formation of a monophasic reaction mixture. Even if the reactant mixture used in the method according to the invention is initially biphasic, this relatively rapidly becomes monophasic in the course of the reaction. If, however, the molar ratio is in the range not in accordance with the invention of less than 1, the reaction mixture is at the beginning initially biphasic for a longer time and becomes monophasic only in the later course of the reaction. The enhanced formation of undesired by-products is associated with the longer duration of biphasicity.

In order to keep the multiple addition of formaldehyde (II) to dialkyl ketone (III) to a level as low as possible, an excess of dialkyl ketone (III) is generally used in the method according to the invention. The molar ratio of dialkyl ketone (III) to formaldehyde (II) in the liquid phase is preferably from 1.5 to 10, particularly preferably 2 and especially preferably ≥3, and preferably ≤7 and particularly preferably ≤5.

The dialkyl ketone (III) to be used in the method according to the invention comprises 3 to 7 carbon atoms and may be branched or unbranched. Unbranched dialkyl ketones (III) include, for example, acetone, methyl ethyl ketone, methyl n-propyl ketone, diethyl ketone (IIIa), methyl n-butyl ketone, ethyl n-propyl ketone, methyl n-pentyl ketone, ethyl n-butyl ketone or di-n-propyl ketone. Examples of branched dialkyl ketones (III) include methyl isopropyl ketone, methyl isobutyl ketone, methyl sec-butyl ketone, ethyl isopropyl ketone, methyl 2-pentyl ketone, methyl 3-pentyl ketone, methyl 2-methylbutyl ketone, methyl 3-methylbutyl ketone or methyl 3-methylbut-2-yl ketone.

An unbranched dialkyl ketone (III) is preferably used, wherein this particularly preferably comprises 4 to 6 carbon atoms. Particular preference is therefore given to methyl ethyl ketone, methyl n-propyl ketone, diethyl ketone (IIIa), methyl n-butyl ketone and ethyl n-propyl ketone. Diethyl ketone (IIIa) may be mentioned as especially preferred dialkyl ketone (III).

The dialkyl ketones (III) to be used are generally very readily accessible by the synthetic routes known in the specialist field. For instance, diethyl ketone (IIIa) can be classically obtained by hydroformylating ethene with carbon monoxide and hydrogen.

By means of the reaction of formaldehyde (II) with the dialkyl ketone (III) specified, an hydroxymethyl group is added (aldol addition) to the position a to the keto group of the dialkyl ketone (III). By means of this so-called aldol addition, the β-hydroxyketone (I) corresponding to the dialkyl ketone (III) used is formed. For instance, 1-hydroxy-2-methyl-3-pentanone (Ia) is formed from formaldehyde (II) and diethyl ketone (IIIa)

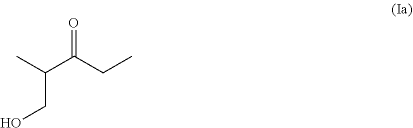

1-Hydroxy-2-methyl-3-pentanone (Ia) is very particularly preferably prepared in the method according to the invention by reacting formaldehyde (II) and diethyl ketone (IIIa).

In the method according to the invention, formaldehyde of various qualities and forms may be used. For instance, it is possible to also feed pure formaldehyde to the reactor. The use of paraformaldehyde is also possible in principle, although not particularly preferred, Typically however, easy to handle formaldehyde sources are used, such as especially its aqueous solutions. These may be of technical-grade quality and laboratory quality. The formaldehyde content of aqueous solutions is typically in the range from 30 to 60% by weight. For instance, in the method according to the invention, the formaldehyde (II) is preferably used in the form of its aqueous solutions having a formaldehyde content of 30 to 60% by weight.

Aqueous solutions of formaldehyde also typically comprise methanol as stabilizer. Since formaldehyde is commonly obtained by catalytic oxidation of methanol, the methanol present is generally wholly or partly unreacted methanol from the formaldehyde synthesis. Depending on the quality of the formaldehyde solution, the methanol content can vary in a wide range. Generally therefore, in the method according to the invention, aqueous formaldehyde solutions are used having a methanol content of 0.1 to 20% by weight, preferably 0.2 to 5% by weight.

In general, an aqueous methanol-containing solution of formaldehyde is used as formaldehyde source in the method according to the invention.

The aldol addition according to the invention is base-catalyzed, specifically by using a trialkylamine having 1 to 4 carbon atoms per alkyl group. The alkyl groups are methyl, ethyl, n-propyl, isopropyl, n-butyl, isobutyl and sec-butyl. The respective n-alkyl radicals are preferably methyl, ethyl, n-propyl and n-butyl. Preferred trialkylamines include trimethylamine, N,N-dimethylethylamine, N,N-diethylmethylamine, triethylamine, N,N-dimethyl-n-propylamine, N-ethyl-N-methyl-n-propylamine, N,N-dimethylisopropylamine, Nethyl-N-methylisopropylamine, N,N-dimethyl-n-butylamine, N, N-dimethylisobutylamine, N, N-dimethylsec-butylamine, tri-n-propylamine and tri-n-butylamine.

Particular preference is given to trialkylamines having a boiling point at 0.1013 MPa abs of less than 102° C., namely trimethylamine, N,N-dimethylethylamine, N, N-diethylmethylamine, triethylamine, N-dimethyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine and N,N-dimethylsec-butylamine. The trialkylamines specified are particularly advantageous when using diethyl ketone (IIIa), since these can be removed as required by distillation as low boilers from diethyl ketone (IIIa). Very particular preference is given to the two trialkylamines having three identical unbranched alkyl groups, namely trimethylamine and triethylamine, especially trimethylamine.

The trialkylamines mentioned are readily accessible by customary synthetic methods, especially trimethylamine and triethylamine.

In addition to the two reactants formaldehyde (II) and dialkyl ketone (III) and the trialkylamine functioning as catalyst, water is also used as further component in the method according to the invention. For instance, the reaction specified is carried out in the presence of 1 to 25% by weight water, based on the liquid phase. This is typically added via the formaldehyde source, i.e. an aqueous formaldehyde solution, via a separate addition of water or in parallel by both possibilities. In the presence of trialkylamine, the presence of water enables the formation of trialkylammonium hydroxide, which is the active part of the catalyst. The amount of water therefore influences the reaction rate. With increasing amount of water, the reaction rate also generally increases. In the context of the invention, however, it has also been shown that, surprisingly, greater amounts of water promote the formation of undesired by-products disproportionately. Thus, the method according to the invention is preferably carried out in the presence of ≥5% by weight, particularly preferably ≥8% by weight and especially preferably ≥10% by weight, and preferably in the presence of ≤17% by weight and particularly preferably ≤15% by weight, based in each case on the liquid phase. In particular, the reaction is carried out in the presence of 10 to 15% by weight water, based on the liquid phase.

If an aqueous methanol-containing formaldehyde solution is used as formaldehyde source in the method according to the invention, the method according to the invention is inevitably also carried out in the presence of methanol. Independently thereof, methanol can however also be added to the reaction separately. Furthermore, it is possible and possibly even advantageous to recycle into the reactor the methanol present in the reaction mixture, together with other components such as unreacted dialkyl ketone (III), water and/or trialkylamine, in the context of the work-up of the reaction mixture.

The method according to the invention is therefore generally carried out in the presence of 0.1 to 10% by weight methanol, based on the liquid phase. In accordance with the invention, it was recognized that methanol contents above 10% by weight markedly slow the reaction. The reason is probably the enhanced formation of acetals from formaldehyde and methanol, which results in loss of freely available formaldehyde. Thus, the method according to the invention is preferably carried out in the presence of ≤6% by weight and particularly preferably ≤3% by weight methanol, and preferably in the presence of ≥0.1% by weight and particularly preferably ≥0.5% by weight methanol, based in each case on the liquid phase.

The method according to the invention is carried out at a temperature of 50 to 150° C., preferably at ≥55° C., particularly preferably at ≥60° C. and especially preferably at ≥65° C., and preferably at ≤125° C. and particularly preferably at ≤100° C. In addition, the method according to the invention is carried out at a pressure of 0.2 to 10 MPa abs, preferably 0.3 MPa abs and particularly preferably ≥0.4 MPa abs, and preferably ≤6 MPa abs, particularly preferably ≤3 MPa abs and especially preferably ≤2 MPa abs.

According to the temperature and pressure parameters specified, the reaction mixture is completely, or at least to a considerable proportion, present in the liquid phase.

The method according to the invention can be performed batchwise, continuously or semi-continuously.

In the batchwise variant, the reactants formaldehyde (II) and dialkyl ketone (III), together with the trialkylamine, water and optionally further components such as methanol for example, are initially charged in the desired amounts in the reactor and these are subsequently brought to the desired temperature and the desired pressure with appropriate mixing of the reaction mixture for the desired reaction time. After the desired reaction time, the reaction mixture is withdrawn for further processing, optionally after completion of depressurization and cooling.

In the continuous variant, the reactants formaldehyde (II) and dialkyl ketone (III), together with trialkylamine, water and optionally further components, such as methanol for example, are fed continuously to the reactor in the desired amounts and, at suitable points, a corresponding amount of reaction mixture is also continuously withdrawn. The reaction mixture is accordingly reacted in the reactor at the desired temperature and the desired pressure. The residence time in the continuous method is dependent on the desired reaction progress. The reaction mixture continuously withdrawn can then be worked-up accordingly.

In the semi-continuous variant, typically dialkyl ketone (III), together with trialkylamine and optionally water and further components, such as methanol for example, are initially charged in the desired amounts in the reactor, these are then brought with appropriate mixing to the desired temperature and the desired pressure and the desired amount of formaldehyde (II) and optionally water and methanol are then fed over a certain time period. After the desired reaction time, the reaction mixture is withdrawn for further processing, optionally after completion of depressurization and cooling.

Since the aldol addition is weakly exothermic, heat of reaction is formed during the method according to the invention. This can be dissipated in adiabatic mode via the reaction output. In isothermal mode of operation, this is already dissipated in the reactor during the reaction and the reaction temperature stays constant. In the method according to the invention, in principle both variants and all in-between stages are possible. In principle, variants are also possible in which, for example, thermal energy is supplied externally and the reaction mixture heats up more intensely than in adiabatic mode, or for example in which more thermal energy is removed than is required for an isothermal mode of operation, the reaction mixture thus being cooled during the reaction.

For the method according to the invention, in principle any apparatuses can be used which are suitable for carrying out reactions in the liquid phase. Non-limiting examples include flow tube, jet loop reactor, stirred tank and stirred tank cascade and combinations thereof. Independently of whether the reaction is conducted in batchwise mode, continuously or semi-continuously, particular reactors have proven to be particularly advantageous. For instance, in a preferred variant, the method is carried out continuously in a flow tube and in another preferred variant, the method is carried out batchwise or semi-continuously in a stirred tank. A combination of one or more stirred tanks or jet loop reactors as preliminary reactor(s) and one or more flow tubes as postreactor(s) is also possible.

In accordance with the selected residence time of the reaction mixture in the reactor, the conversion of the reaction mixture can be adjusted. At a fairly low residence time of less than one hour, the conversion is generally incomplete such that all of the formaldehyde (II) was typically still not reacted and is subsequently to be removed. Conversely, if the residence time is close to one day or even longer, the amount of undesired by-products also generally increases. In addition, a long residence time also reduces the space-time yield.

In the batchwise process, the reactor is thus typically left at the desired reaction conditions for 1 to 16 hours, preferably 1.5 and particularly preferably ≥2 hours, and preferably ≤10 and particularly preferably ≤6 hours.

Accordingly, in the continuous process, the flow volume entering the reaction system is adjusted so that a residence time results of typically 1 to 16 hours, preferably ≥1.5 and particularly preferably ≥2 hours, and preferably ≤10 and particularly preferably ≤6 hours at the desired reaction conditions.

The semi-continuous process is modelled on the batchwise process with respect to the reaction time.

The reaction mixture obtained by the reaction according to the invention in particular comprises the β-hydroxyketone (I) formed, unreacted dialkyl ketone (III), trialkylamine, water and by-products formed, such as di- and trihydroxy compounds formed by multiple addition for example, and possibly even enones formed already by dehydration. In the case where methanol was present in the reaction, the reaction mixture also comprises methanol. To obtain β-hydroxyketone (I) in the highest possible purity, it is expedient to work-up the reaction mixture accordingly. In this case, a multi-stage distillative work-up is particularly useful in which components lighter boiling than the β-hydroxyketone (I) and the components higher boiling than the β-hydroxyketone (I) are removed.

In the method according to the invention, a work-up is preferred in which the components lighter boiling than the β-hydroxyketone (I) are removed by distillation from the output of the reactor and purified β-hydroxyketone (I) is obtained as low boiler by distillation from the bottom product containing β-hydroxyketone (I). The variant specified therefore comprises at least two distillation columns connected in series. As an alternative, it is also possible to use a dividing wall column. Advantageously, the components relevant for the reaction, such as unreacted dialkyl ketone (III) and trialkylamine after removal thereof in the distillative work-up specified, are fed back to the reactor.

In a preferred variant, the distillative removal of the components lighter boiling than the β-hydroxyketone (I) is carried out in two distillation columns connected in series, in which the trialkylamine and the unreacted dialkyl ketone (III) are removed separately. Depending on the position of the boiling points of these two components, firstly the trialkylamine and subsequently the dialkyl ketone (III) are removed or vice versa. As a result, it is possible for example to recycle the two components separately to the reactor.

If methanol is present in the reaction mixture, this also arises as a light boiler in the purification described. In order to counteract an accumulation of methanol in the reactor, it is advantageous to discharge at least some of the methanol from the system as so-called purge stream. The specific discharge of methanol can be effected, for example in the case of a trialkylamine lower boiling than dialkyl ketone (III), as a mixture with trialkylamine, or in the case of a trialkylamine higher boiling than dialkyl ketone (III), as a mixture with dialkyl ketone (III) at the top of the first column or by a further distillative separation of the low boilers obtained in the purification described or optionally facilitated by stripping of the low boilers obtained.

It is likewise advantageous to discharge at least some of the water from the system as further so-called purge stream in order also here to counteract an accumulation. The specific discharge of water can be effected, for example, by a further distillative separation of the low boilers obtained in the purification described. Depending on the type of dialkyl ketone (III) used and the trialkylamine, separated low boiler products can optionally be separated into an organic and an aqueous phase. In such a case, it is then convenient to separate off and completely or at least partially discharge the aqueous phase.

Particularly advantageous is a method for preparing 1-hydroxy-2-methyl-3-pentanone (Ia) by reacting formaldehyde (II) with diethyl ketone (IIIa), in which
(a) trimethylamine or triethylamine is used as trialkylamine,
(b) the reaction is carried out in the presence of 0.1 to 6% by weight methanol, based on the liquid phase,
(c) the output of the reactor is fed to a first distillation column and the trialkylamine and methanol used are removed therefrom as low boilers by distillation,
(d) the bottoms output of the first distillation column is fed to a second distillation column and diethyl ketone (IIIa) and water are removed therefrom as low boilers by distillation, and
(e) the bottoms output of the second distillation column is fed to a third distillation column and purified 1-hydroxy-2-methyl-3-pentanone (Ia) is obtained therefrom as low boiler by distillation.

Since methanol and also trimethylamine and triethylamine have a lower boiling point than diethyl ketone (IIIa), it is possible to already remove the components specified in step (c) before diethyl ketone (IIIa) and water are separated in step (d). As an alternative to trimethylamine and triethylamine, it is also possible however to use in this variant the mixed trialkylamines N,N-dimethylethylamine, N,N-diethylmethylamine, N,N-dimethyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine and N,N-dimethyl-sec-butylamine, each having a boiling point below the boiling points of diethyl ketone (IIIa) and water.

Separate recovery of a low boiler product comprising trialkylamine and methanol from step (c) and a low boiler product comprising diethyl ketone (Ilia) and water from step (d) enables a more effective as well as more efficient reuse of relevant components in the case of simultaneous discharge of excess components.

Thus, it is, for example, possible to separate off some of the methanol from the low boiler product of step (c) comprising the trialkylamine and methanol and to discharge it as so-called purge stream, to feed back a certain portion to the distillation column as return and to recycle the remaining stream to the reactor.

In a preferred variant using trimethylamine as trialkylamine, which is lower boiling than methanol, preferably methanol is condensed in a first condenser from the low boiler product removed from the column of step (c), a portion of this condensed stream is discharged as purge stream and the other portion is fed back to the column as return. The non-condensed portion from the first condenser is led to a second condenser in order to condense trimethylamine and the condensed stream is fed back to the reactor as recycle stream.

In another preferred variant using trimethylamine as trialkylamine, which is lower boiling than methanol, the desired amount of methanol as purge stream is withdrawn as sidestream from the rectifying section of the column in step (c). Trimethylamine is obtained via the overhead as low boiler product and this is fed to a condenser in order to condense trimethylamine. A portion of the condensed stream is fed back as return to the column and the remaining portion is fed back to the reactor as recycle stream.

In a further preferred variant using trimethylamine as trialkylamine, which is lower boiling than methanol, a dividing wall column is used in step (c). Trimethylamine is obtained in this case via the overhead as low boiler product and this is fed to a condenser in order to condense trimethylamine. A portion of the condensed stream is fed back as return to the column and the remaining portion is fed back to the reactor as recycle stream. The desired amount of methanol as purge stream is withdrawn as sidestream.

In a preferred variant, using triethylamine as trialkylamine, which is higher boiling than methanol, the low boiler product removed from the column of step (c) is substantially completely condensed in a condenser, a portion of the condensed stream is discharged as purge stream, a further portion of the condensed stream is fed back to the column as return and the residual portion of the condensed stream is fed back to the reactor as recycle stream.

In another preferred variant using triethylamine as trialkylamine, which is higher boiling than methanol, triethylamine is withdrawn as sidestream from the rectifying section of the column in step (c) and this is fed back to the reactor as recycle stream. Methanol is obtained as top stream, which is condensed, the desired amount is discharged as purge stream and the remaining stream is fed back to the column as return.

In a further preferred variant using triethylamine as trialkylamine, which is higher boiling than methanol, a dividing wall column is used in step (c). Methanol is obtained in this case via the overhead as low boiler product and this is fed to a condenser in order to condense methanol. The desired amount is discharged from the condensate as purge stream and the remaining stream is fed back to the column as return. Triethylamine is withdrawn as sidestream and fed back to the reactor as recycle stream.

In a further preferred variant using triethylamine as trialkylamine, which is higher boiling than methanol, triethylamine is already removed separately in the form of an upper sidestream from the column in step (c), which is condensed and the condensed stream fed back to the reactor as recycle stream. The top stream removed therefore predominantly comprises methanol. After condensation thereof, a portion of the condensed stream is discharged as purge stream and the remaining portion is fed back to the column as return.

For instance, it is particularly advantageous in step (c) to feed 10 to 100% by weight, 50 to 100% by weight, primarily 90 to 100% by weight, particularly 95 to 100% by weight and especially 99 to 100% by weight of the separated trialkylamine back to the reactor.

Furthermore, it is possible, for example, to remove water from the low boiler product comprising diethyl ketone (IIIa) and water of step (d) and to discharge it as further so-called purge stream, and to feed the remaining stream comprising diethyl ketone (IIIa) back to the reactor. Thus, it is particularly advantageous if the low boiler product removed in step (d) is separated into an aqueous and an organic liquid phase, comprising diethyl ketone (IIIa), and the organic liquid phase comprising diethyl ketone (IIIa) is fed back to the reactor to an extent of 50 to 100% by weight, preferably 90 to 100% by weight, particularly 95 to 100% by weight and especially 99 to 100% by weight.

In a further variant, it is also possible to recycle some of the aqueous liquid phase to the reactor as water source.

When obtaining purified 1-hydroxy-2-methyl-3-pentanone (Ia) as low boiler in step (e), a high-boiler stream is formed as bottoms product, which primarily comprises the by-products formed during the reaction and the subsequent distillative work-up. This stream can be discharged from the system and be used for thermal purposes, for example.

As mentioned at the outset, di- and trihydroxy compounds are formed in the method according to the invention by multiple addition of formaldehyde (II) to dialkyl ketone (III). These may dehydrate to form enones. The latter may in turn dimerize, oligomerize or even polymerize and thus form high-boiling components. The latter are concentrated in the bottoms product when obtaining the purified β-hydroxyketone (I) and can therefore be readily discharged from the system.

In the case of the production of 1-hydroxy-2-methyl-3-pentanone (Ia) by reacting formaldehyde (II) with diethyl ketone (IIIa), the di- and trihydroxy compounds, inter alia, form as by-products.

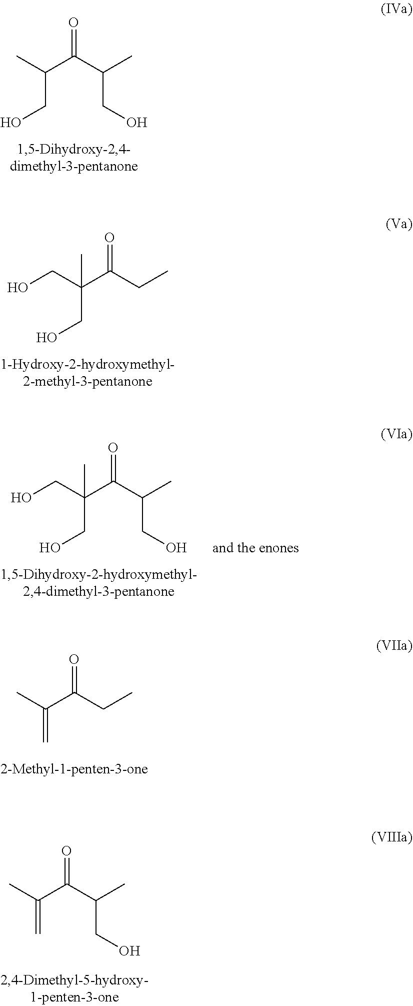

By dimerization of 2-methyl-1-penten-3-one (VIIa), the enone dimer (IXa) can form, which is a high boiler compared to 1-hydroxy-2-methyl-3-pentanone (Ia).

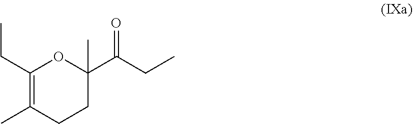

It may be mentioned that the enone (VIIIa) in particular, due to its boiling point, can only be separated by distillation with relative difficulty from 1-hydroxy-2-methyl-3-pentanone (Ia). For instance, 1-hydroxy-2-methyl-3-pentanone (Ia) boils at 0.002 MPa abs at approximately 95° C. for example, whereas the enone (VIIIa) at 0.002 MPa abs only boils at around 10° C. higher at approximately 105° C. Therefore, in the distillative work-up to obtain purified 1-hydroxy-2-methyl-3-pentanone (Ia), a small portion of the enone (VIIIa) together with the 1-hydroxy-2-methyl-3-pentanone (Ia) is removed as low-boiler stream. A more exhaustive purification would require a multiple distillation with corresponding loss of yield or necessitate the use of a more complex distillation column with greater separation efficiency.

Thus, for example, in the preparation of 1-hydroxy-2-methyl-3-pentanone (Ia) in particular, a reduced formation of the enone (VIIIa), which the method according to the invention enables, is particularly advantageous.

By means of distillative purification of the reaction mixture formed in the reaction according to the invention, with removal by distillation of the components lighter boiling than β-hydroxyketone (I) and subsequent separation by distillation of the β-hydroxyketone (I) as low boiler from the bottoms product previously obtained, β-hydroxyketone (I) can be obtained with a purity above 90% by weight.

In a general embodiment of the method according to the invention for batchwise preparation, the reactants dialkyl ketone (III) and formaldehyde (II) and also trialkylamine and water, which also may optionally originate completely from an aqueous formaldehyde solution, and the molar ratio of trialkylamine to formaldehyde (II) according to the invention, are initially charged in a pressure-resistant stirred tank or autoclave and adjusted to the desired pressure and temperature reaction parameters by thorough mixing. After the desired reaction time, the reaction mixture is cooled and depressurized. This may be effected for example in the reaction apparatus used or by removal of the reaction mixture therefrom. To isolate the β-hydroxyketone (I) product of value, the reaction mixture can be subsequently worked-up. The work-up is preferably achieved by multi-stage distillation, in which initially, in one or two distillation stages, diverse low boilers such as water, trialkylamine, unconverted reactants, primarily dialkyl ketone (III), and also low-boiling by-products are removed. The trialkylamine and dialkyl ketone (III) removed can be reused in a further batch as starting material. The mixture freed of low boilers is then fed to a further distillation column for the isolation of β-hydroxyketone (I). High boilers are removed via the bottoms and the β-hydroxyketone (I) is withdrawn as side product or overhead product.

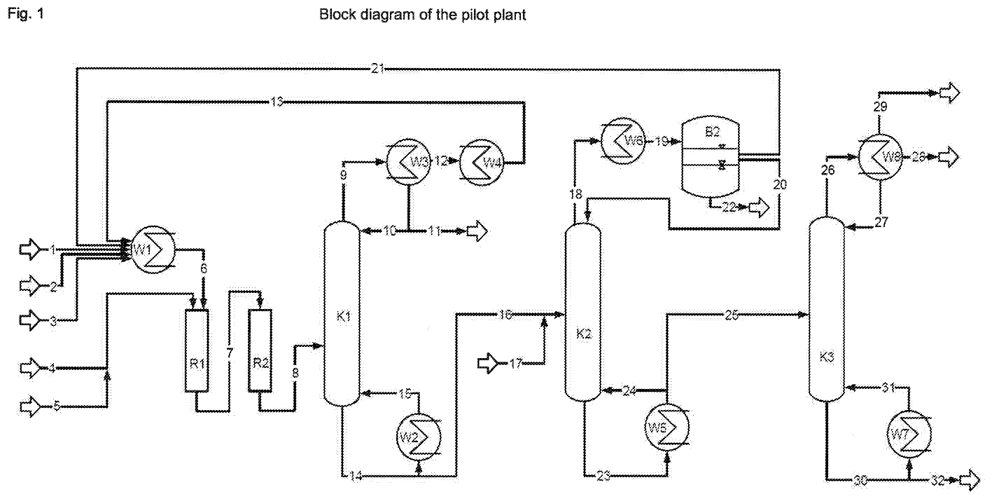

In a general embodiment of the method according to the invention for continuous preparation, the reactants dialkyl ketone (III) and formaldehyde (II) and also trialkylamine and water, which also may optionally originate completely from an aqueous formaldehyde solution, in the molar ratio of trialkylamine to formaldehyde (II) according to the invention, are fed continuously to a flow tube at the desired pressure and the desired temperature. The reaction takes place therein. The geometry and the flow rate are assessed so that the residence time corresponds to the desired reaction duration. The reaction mixture obtained can then be worked-up in analogy to the work-up described in the batchwise process. An exemplary representation of a continuously operated plant is shown in FIG. 1. A further description for this purpose is found below in the description of the pilot plant.

The method according to the invention enables the preparation of β-hydroxyketones in high selectivity, yield and purity using readily accessible feedstocks. It is simple, safe and efficient to conduct in common apparatuses and enables a high space-time yield therein. In particular, the method according to the invention is characterized by a distinctly reduced formation of undesired by-products. Therefore, the feedstocks can be used more purposefully for the synthesis of the desired β-hydroxyketones and the complexity for removing and disposal of undesired by-products is reduced.

### EXAMPLES

The invention was tested experimentally both in a pilot plant and in a laboratory-scale plant.

### Pilot Plant

The simplified block diagram of the pilot plant is shown in FIG. 1. The individual apparatus labels have the following meanings therein:

W1: Heat exchanger (heated with thermostat)
R1, R2; Tubular reactors (reaction volumes 8.5 L each)
K1: Bubble-cap tray column (□55 mm, 45 trays)
W2: Falling-film evaporator (0.45 m², heated with thermostat)
W3: Condenser, 0.17 m² (cooled with cryostat)

### W4: Condenser (0.16 m²)

K2: Column (□50 mm, Montz A3-750 packings, height 2560 mm)
W5: Falling-film evaporator (0.4 m², heated with thermostat)
W6: Condenser, (0.2 m², cooled with cryostat)
B2: Phase separator (0.15 L)
K3: Column (□43 mm, Montz A3-750 packings, height 2020 mm)
W7 Falling-film evaporator (0.4 m², heated with thermostat)
W8: Condenser, (0.2 m², cooled with cryostat)

Diethyl ketone (stream (1)), trimethylamine (stream (2)) and methanol (stream (3)) and the recycle streams (stream (13) and stream (21)) were fed to the heat exchanger W1 and subsequently passed into the tubular reactor R1 via stream (6). W1 served to adjust the inlet temperature of R1. In parallel thereto, the aqueous formaldehyde solution (stream (4)) and water (stream (5)) were passed into R1 together as separate streams. The reaction mixture from R1 was fed via stream (7) to tubular reactor R2. Both reactors were operated adiabatically. In the reactors, formaldehyde and diethyl ketone were reacted to give 1-hydroxy-2-methyl-3-pentanone and diverse by-products.

The reaction mixture from R2 was fed via stream (8) to column K1 and trimethylamine and methanol were distilled off therein via the overhead. In condenser W3, as much distillate was condensed so that in each case the return provided via stream (10) and the purge stream (11) required to set a constant methanol concentration was formed. In condenser W4, the residual vapors, which predominantly comprised trimethylamine, were condensed and fed back to the heat exchanger W1 via stream (13). In the falling-film evaporator W2, the temperature of the bottom stream was adjusted so that the methanol concentration in the bottom of column K1 was at most 0.1% by weight.

The bottoms output from column K1 was fed to column K2 as stream (16), wherein stabilizer (phenothiazine) was metered in via stream (17). In column K2, unreacted diethyl ketone and water were distilled off via the overhead. The vapor stream (18) formed was condensed in this case in condenser W6 and then passed into the phase separator B2. The condensate was separated therein into a more dense aqueous and a less dense organic phase. The aqueous phase was disposed of via stream (22). The organic phase, which comprised predominantly diethyl ketone, was passed back via stream (20) to adjust the desired return to column K2. The excess was fed back to the heat exchanger W1 via stream (21). In the falling-film evaporator W5, the temperature of the bottom stream was adjusted so that the diethyl ketone concentration in the outlet of falling-film evaporator W5 was approximately 0.1% by weight.

The bottoms output from column K2 was fed to column K3 via stream (25). In column K3, the 1-hydroxy-2-methyl-3-pentanone (Ia) formed and also enone (VIIa) was distilled off via the overhead. The vapor stream (26) formed was subsequently condensed in condenser W8 by an appropriate temperature adjustment to the extent that 1-hydroxy-2-methyl-3-pentanone (Ia) could be withdrawn having only a low concentration of enone (VIIa) as stream (28). A portion of the condensate was fed back again to column K3 as return stream (27). The uncondensed enone (VIIa)-containing vapors were withdrawn via stream (29). In the falling-film evaporator W7, the temperature of the bottom stream was adjusted so that the concentration of enone (VIIIa) in the 1-hydroxy-2-methyl-3-pentanone (Ia)-containing product stream (28) was in the desired range. The high boilers separated were withdrawn via stream (32).

### Laboratory Plant

A 2.5 L thermostatically controllable steel autoclave served as laboratory plant equipped with stirrer, thermocouple, pressure sensor and metered addition and withdrawing tube.

### General Operation of the Pilot Plant

In examples 1 and 2, the following fresh feeds were fed to the pilot plant:
Stream (1): Diethyl ketone (“DEK”)
Stream (2): Trimethylamine (“TMA”)
Stream (3): Methanol (“MeOH”)
Stream (4): aqueous formaldehyde solution (“FA solution”) having a formaldehyde content of 30% by weight (“FA30”) and in each case ca. 1% by weight methanol content
Stream (5): Water
Stream (17): Solution of 10% by weight phenothiazine in diethyl ketone (“Stabi-solution”)

The 1-hydroxy-2-methyl-3-pentanone (“HMP”) formed and purified by distillation was withdrawn via stream (28).

### General Operation of the Laboratory Plant

In examples 3 to 9, in each case diethyl ketone, aqueous formaldehyde (methanol-free), water and methanol were initially charged in the steel autoclave. The autoclave was sealed, purged with nitrogen, pressurized to 0.5 MPa abs nitrogen and heated to the desired temperature with stirring (900 rpm). By means of a screw press pump, the desired amount of trialkylamine was added within a few minutes. Subsequently, the autoclave was further stirred at autogenous pressure and maintaining the desired temperature constant and, at certain time intervals, about 3-5 g samples were withdrawn in each case into a sample vial. The samples withdrawn were immediately cooled down in an ice bath in order to prevent further reaction and then intermediately stored in the refrigerator until analysis by gas chromatography. At the end of the experiment, the autoclave was then cooled to ca. 40° C., depressurized, outgassed for 2 hours, cooled to room temperature and finally emptied.

### Determination of the Phase Nature

The phase nature of the reaction mixture was determined both in the operation of the pilot plant and in the operation of the laboratory plant. For this purpose, the reaction mixture was withdrawn at the outlet of the tubular reactors R1 and R2 during operation of the pilot plant, and via the withdrawal tube during operation of the laboratory plant and, after a brief waiting time in the low minute range, visually examined. The sample was then cooled down in each case in the ice bath for interim storage for the subsequent analysis by gas chromatography.

### Quantitative Analysis

The quantitative analysis of the reaction mixture was conducted by gas chromatography (ZB-1 column, 30 m, 1 μm, 0.25 mm). In the case of a biphasic sample, this was firstly homogenized with ethanol and the ethanol peak subsequently deducted. Diethylene glycol diethyl ether served as internal standard for all quantitative GC analyses. By means of the response factors experimentally determined beforehand on the pure substances, methanol, diethyl ketone (IIIa) and 1-hydroxy-2-methyl-3-pentanone (Ia) were determined quantitatively. For 1,5-dihydroxy-2,4-dimethyl-3-pentanone (IVa), 1-hydroxy-2-hydroxymethyl-2-methyl-3-pentanone (Va), 1,5-dihydroxy-2-hydroxymethyl-2,4-dimethyl-3-pentanone (VIa), 2-methyl-1-penten-3-one (VIIa) and 2,4-dimethyl-5-hydroxy-1-penten-3-one (VIIIa), the response factors were calculated on the basis of the “effective carbon number concept”.

### Examples 1 to 2

In examples 1 and 2, the pilot plant was operated as presented in Table 1. In example 2, only the two tubular reactors R1 and R2 were operated. Table 2a accounts for the components diethyl ketone (IIIa), formaldehyde (II), water, methanol and trimethylamine fed to the reactor R1 based on Table 1. This shows that in example 1 a molar ratio according to the invention of trimethylamine to formaldehyde (II) of 1.75 was used. In example 2, the corresponding molar ratio was only 0.23.

Owing to this low molar ratio, the output of reactor R1 in example 2 was biphasic, whereas in inventive example 1 the output of R1 was monophasic. Only by passing through reactor R2 were the outputs monophasic in both examples. A comprehensive summary is found in Table 2b.

Table 2c shows the analytical data of the outputs of reactor R2 of both examples for the following components specified:

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

In addition to these, the outputs naturally comprise further components such as unreacted diethyl ketone (IIIa), water, trimethylamine, methanol and also further by-products. A comparison of the analyses of example 1 and 2 shows that, in comparative example 2, at a low molar ratio of trimethylamine to formaldehyde (II) in the inlet of reactor R1 of only 0.23, significantly more primary by-products (IVa), (Va) and (VIa) are formed than in the inventive example 1 at a high molar ratio of 1.75. For instance, the output of reactor R2 of comparative example 2 comprises an approximately 28% higher amount of the components (IVa) and (Va) formed by two-fold addition, at 3.7% by weight, than the output of reactor R1 of inventive example 1 at only 2.9% by weight. More significant is the difference in component (VIa) formed by three-fold addition. Here, approximately 267% more component (VIa) was formed in example 2, at 1.1% by weight, than at only 0.3% by weight in example 1. Correspondingly, the total content of by-products (IVa) to (VIIIa) is also significantly higher in example 2 than in example 1.

After the three-stage distillative work-up, the desired target product 1-hydroxy-2-methyl-3-pentanone (Ia) is obtained at a purity of 93.7% by weight. Selected analytical data of the pure product removed as stream 28 are shown in Table 2d. Main impurities are the by-products (VIIa) and (VIIIa) formed predominantly in the distillative work-up by thermally induced dehydration which, due to their boiling points, can only be removed with relative difficulty from 1-hydroxy-2-methyl-3-pentanone (Ia) by distillation.

Example 1 proves that using trimethylamine as basic component and a molar ratio of trimethylamine to formaldehyde (II) of 1.75 in the reaction of diethyl ketone (IIIa) with formaldehyde (II) by the method according to the invention, substantially fewer undesired by-products are formed than in comparative example 2 at a corresponding molar ratio of only 0.23.

### Examples 3 to 5

Examples 3 to 5 were conducted in the laboratory-scale plant and show the influence of water on the reaction process. The respective method conditions are given in Tables 3a, 4a and 5a. During the experiment, several samples were taken in each case over a period of 0.5 to 12 hours, which were tested with respect to their phase nature and analyzed by gas chromatography The values obtained are shown in Tables 3b, 4b and 5b.

At the start of the reaction, the samples and thus the reaction mixture were in each case biphasic. In the course of the reaction, the reaction mixture then became monophasic in the time window of 0.5 to 2 hours reaction time.

Table 6 compares the contents of the components

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one
and the sum total of (IVa) to (VIIIa) after a reaction time of 5 hours depending on the amount of water. The tables indicate that, at a relatively high water content of 19% by weight at the start of the reaction, the amount of by-products (IVa) to (VIIIa) formed has significantly increased. For instance, at a water content at the start of the reaction of 19% by weight, in total already 5.26% by weight of by-products (IVa) to (VIIIa) are formed, whereas at a water content at the start of the reaction of 10% by weight or 14% by weight, in total only 4.25% by weight and 3.77% by weight are formed respectively. At all three water contents, the content of target product 1-hydroxy-2-methyl-3-pentanone (Ia) is in the range from 12.55 to 13.83% by weight with no discernible trend.

Examples 3 to 5 show that even at a water content of 19% weight, a sufficiently high amount of target product 1-hydroxy-2-methyl-3-pentanone (Ia) is formed, but at the same time the formation of by-products (IVa) to (VIIIa) increases.

### Examples 6 to 8

Examples 6 to 8 were conducted in the laboratory-scale plant and show, together with example 4, the influence of methanol on the reaction process. The method conditions of examples 6 to 8 are given in Tables 7a, 8a and 9a. During the experiment, several samples were taken in each case over a period of 0.5 to 5 hours (in example 4 up to 12 hours), which were tested with respect to their phase nature and analyzed by gas chromatography. The values obtained are shown in Tables 4b, 7b, 8b and 9b.

At the start of the reaction, the samples and thus the reaction mixture were in each case biphasic. In the course of the reaction, the reaction mixture then became monophasic in the time window of 0 to 1 hour reaction time.

Table 10 compares the contents of the components

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-penta none
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one
and the sum total of (IVa) to (VIIIa) after a reaction time of 5 hours depending on the amount of methanol. The tables indicate that, at a methanol content in the range from 0.5 to 6% by weight, the target product 1-hydroxy-2-methyl-3-pentanone (Ia) is formed in sufficiently high amount and, at increasing methanol content, the sum of by-products (IVa) to (VIIIa) here even decreases.

### Example 9

Example 9 was likewise conducted in the laboratory-scale plant and shows, together with example 5, the influence of the reaction temperature on the reaction process. The method conditions of example 9 are shown in Table 11a. During the experiment, several samples were taken in each case over a period of 0.5 to 5 hours (in example 5 up to 12 hours), which were tested with respect to their phase nature and analyzed by gas chromatography. The values obtained are shown in Tables 5b and 11b.

At a reaction temperature of 70° C., the reaction mixture was only monophasic after a reaction time in the range of 2-3 hours, whereas in example 5, at 90° C., the reaction mixture was already monophasic after a reaction time in the range of 0.5-1 hour.

Table 12 compares the contents of the components

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one
and the sum total of (IVa) to (VIIIa) after a reaction time of 5 hours depending on the reaction temperature, it is evident from the tables that the formation of the target product 1-hydroxy-2-methyl-3-pentanone (Ia), but also of the by-products (IVa) to (VIIIa) increases with increasing reaction temperature.

TABLE 1
Operation of the pilot plant (examples 1 and 2)

| | | | | Example | |
|---|---|---|---|---|---|
| Apparatus | Description | Stream | Unit | 1 | 2 |
| | DEK | 1 | kg/h | 0.415 | 2.05 |
| | TMA | 2 | kg/h | 0.07 | 0.07 |
| | MeOH | 3 | kg/h | 0.032 | 0.027 |
| | FA solution | 4 | kg/h | 0.521 (FA30) | 0.519 (FA30) |
| | Water | 5 | kg/h | 0.051 | 0.051 |
| | TMA Recycle | 13 | kg/h | 0.49 (4.8% by weight | 0 |
| | | | | MeOH) | |
| | DEK Recycle | 21 | kg/h | 1.641 (2.2% by weight water) | 0 |
| R1 | T(in) | 4 + 5 + 6 | ° C. | 84 | 81.8 |
| | p(in) | 4 + 5 + 6 | MPa abs | 0.75 | 0.75 |
| R2 | T(in) | 7 | ° C. | 89.3 | 86.6 |
| K1 | p(head) | 9 | MPa abs | 0.1023 | — |
| | return | 10 | kg/h | 3.326 | — |
| W2 | T | 15 | ° C. | 87.1 | — |
| K2 | p(head) | 18 | MPa abs | 0.022 | — |
| | return | 20 | kg/h | 2.5 | — |
| | Stabi solution | 17 | kg/h | 0.001 | — |
| W5 | T | 24 | ° C. | 140.2 | — |
| W6 | Condensate | 19 | ° C. | 34.7 | — |
| K3 | p(head) | 26 | MPa abs | 0.0029 | — |
| | return | 27 | kg/h | 1.6 | — |
| W7 | T | 31 | ° C. | 150 | — |
| W8 | T | 28 | ° C. | 80 | — |
| | HMP | 28 | kg/h | 0.412 | — |

TABLE 2a
Reactor R1 input (selected components)

| | | | | | Water | | MeOH | | | | Molar ratio |
|---|---|---|---|---|---|---|---|---|---|---|---|
| Exam- | (IIIa) | | (II) | | | [% by | | [% by | TMA | | TMA/[(II)] |
| ple | [g/h] | [mol/h] | [g/h] | [mol/h] | [g/h] | wt.] | [g/h] | wt.] | [g/h] | [mol/h] | [mol/mol] |
| 1 | 415 + | 23.4 | 156.3 | 5.20 | 359.5 + | 13.9 | 32.0 + | 1.89 | 70.0 + | 9.08 | 1.75 |
| | 1605 = | | | | 51.0 + | | 5.21 + | | 466.5 = | | |
| | 2020 | | | | 3.61 = | | 23.5 = | | 536.5 | | |
| | | | | | 446.6 | | 60.7 | | | | |
| 2 | 2050 | 23.8 | 155.7 | 5.18 | 358.1 + | 15.1 | 27.0 + | 1.19 | 70.0 | 1.18 | 0.23 |
| | | | | | 51.0 = | | | | | | |
| | | | | | 409.1 | | 5.19 = | | | | |

(Ia) 1-Hydroxy-2-methyl-3-pentanone
(II) Formaldehyde
(IIIa) Diethyl ketone

TABLE 2b
Phase nature of reactor outputs

| Example | R1 output | R2 output |
|---|---|---|
| 1 | Monophasic | Monophasic |
| 2 | Biphasic | Monophasic |

TABLE 2c
Reactor R2 output (selected components)

| | | | | | | (IVa) + (Va) + (VIa) + |
|---|---|---|---|---|---|---|
| Exam- | (Ia) | (IVa) + (Va) | (VIa) | (VIIa) | (VIIIa) | (VIIa) + (VIIIa) |
| ple | [% by wt.] | [% by wt.] | [% by wt.] | [% by wt.] | [% by wt.] | [% by wt.] |
| 1 | 12.5 | 2.9 | 0.3 | 0.4 | 0.3 | 3.9 |
| 2 | 11.5 | 3.8 | 1.1 | 0.4 | 0.4 | 5.7 |

TABLE 2d
Purity of (Ia) after distillative work-up (stream 28)

| | (Ia) | (IVa) + (Va) | (VIa) | (VIIa) | (VIIIa) | Water |
|---|---|---|---|---|---|---|
| Exam- | [% by | [% by | [% by | [% by | [% by | [% by |
| ple | wt.] | wt.] | wt.] | wt.] | wt.] | wt.] |
| 1 | 93.7 | 0.0 | 0.0 | 1.5 | 2.8 | 0.1 |

(Ia) 1-Hydroxy-2-methyl-3-pentanone
(IVa) 1,5-Dihydroxy-2.4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 3a
Feedstocks (example 3)

| Starting | | | | | | | |
|---|---|---|---|---|---|---|---|
| weight | | | Mass | | Mass fraction | Molar amount | Reaction |
| [g] | Feedstock | | [g] | | [% by wt.] | [mol] | temperature |
| 768.1 | Diethyl ketone (IIIa) | | 768.1 | | 66.8 | 8.92 | 90° C. |
| 145.7 | Formaldehyde (49%) | Formaldehyde (II) | 71.4 | | 6.2 | 2.38 | |
| | | Water | 74.3 | 115.0 | 10.0 | 6.38 | |

| | | | |
|---|---|---|---|
| 40.7 | Water | 40.7 | Molar ratio |

| | | | | | |
|---|---|---|---|---|---|
| 34.7 | Methanol | 34.7 | 3.0 | 1.08 | TMA/[(II)] |
| 161.0 | Trimethylamine (“TMA”) | 161.0 | 14.0 | 2.72 | 1.14 |

TABLE 3b
Reaction profile (example 3)

| Reaction | | (Ia) | (IVa) + | (VIa) | (VIIa) | (VIIIa) |
|---|---|---|---|---|---|---|
| time | | [% by | (Va) | [% by | [% by | [% by |
| [hours] | Phase nature | wt.] | [% by wt.] | wt.] | wt.] | wt.] |
| 0 | Biphasic | 0 | 0 | 0 | 0 | 0 |
| 0.5 | Biphasic | 6.66 | 0.82 | 0.05 | 0.08 | 0.05 |
| 1 | Biphasic | 8.54 | 1.18 | 0.06 | 0.12 | 0.06 |
| 2 | Monophasic | 10.90 | 1.94 | 0.17 | 0.21 | 0.12 |
| 3 | Monophasic | 12.29 | 2.48 | 0.27 | 0.28 | 0.16 |
| 5 | Monophasic | 13.83 | 3.18 | 0.42 | 0.40 | 0.25 |
| 7 | Monophasic | 14.87 | 3.58 | 0.52 | 0.49 | 0.32 |
| 9 | Monophasic | 15.00 | 3.75 | 0.57 | 0.55 | 0.37 |
| 12 | Monophasic | 15.12 | 3.86 | 0.57 | 0.64 | 0.45 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 4a
Feedstocks (example 4)

| Starting | | | | | Mass | Molar | |
|---|---|---|---|---|---|---|---|
| weight | | | Mass | | fraction | amount | Reaction |
| [g] | Feedstock | | [g] | | [% by wt.] | [mol] | temperature |
| 736.0 | Diethyl ketone (IIIa) | | 736.0 | | 64.0 | 8.55 | 90° C. |
| 117.2 | Formaldehyde | Formaldehyde (II) | 57.4 | | 5.0 | 1.91 | Molar ratio |
| | (49%) | Water | 59.8 | 161.0 | 14.0 | 8.93 | TMA[(II)] |

| | | | |
|---|---|---|---|
| 101.2 | Water | 101.2 | 1.42 |

| | | | | |
|---|---|---|---|---|
| 34.5 | Methanol | 34.5 | 3.0 | 1.08 |
| 161.0 | Trimethylamine (“TMA”) | 161.0 | 14.0 | 2.72 |

TABLE 4b
Reaction profile (example 4)

| Reaction | | | | | | |
|---|---|---|---|---|---|---|
| time | Phase | (Ia) | (IVa) + (Va) | (VIa) | (VIIa) | (VIIIa) |
| [hours] | nature | [% by wt.] | [% by wt.] | [% by wt.] | [% by wt.] | [% by wt.] |
| 0 | Biphasic | 0 | 0 | 0 | 0 | 0 |
| 0.5 | Biphasic | 7.14 | 1.16 | 0.11 | 0.11 | 0.06 |
| 1 | Monophasic | 8.80 | 1.51 | 0.15 | 0.15 | 0.07 |
| 2 | Monophasic | 10.40 | 2.07 | 0.31 | 0.23 | 0.11 |
| 3 | Monophasic | 11.57 | 2.41 | 0.38 | 0.29 | 0.15 |
| 5 | Monophasic | 12.55 | 2.72 | 0.46 | 0.38 | 0.21 |
| 7 | Monophasic | 12.90 | 2.83 | 0.48 | 0.45 | 0.25 |
| 9 | Monophasic | 12.92 | 2.84 | 0.49 | 0.45 | 0.25 |
| 12 | Monophasic | 12.97 | 2.82 | 0.49 | 0.59 | 0.33 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 5a
Feedstocks (example 5)

| Starting | | | | | Mass | Molar | |
|---|---|---|---|---|---|---|---|
| weight | | | Mass | | fraction | amount | Reaction |
| [g] | Feedstock | | [g] | | [% by wt.] | [mol] | temperature |
| 673.4 | Diethyl ketone (IIIa) | | 673.4 | | 58.6 | 7.82 | 90° C. |
| 127.7 | Formaldehyde | Formaldehyde (II) | 62.6 | | 5.4 | 2.08 | Molar ratio |
| | (49%) | Water | 65.1 | 218.5 | 19.0 | 12.1 | TMA[(II)] |

| | | | |
|---|---|---|---|
| 153.4 | Water | 153.4 | 1.31 |

| | | | | |
|---|---|---|---|---|
| 34.5 | Methanol | 34.5 | 3.0 | 1.08 |
| 161.0 | Trimethylamine (“TMA”) | 161.0 | 14.0 | 2.72 |

TABLE 5b
Reaction profile (example 5)

| | | | (IVa) + | | | |
|---|---|---|---|---|---|---|
| Reaction | | (Ia) | (Va) | (VIa) | (VIIa) | (VIIIa) |
| time | Phase | [% | [% | [% | [% | [% |
| [hours] | nature | by wt.] | by wt.] | by wt.] | by wt.] | by wt.] |
| 0 | Biphasic | 0 | 0 | 0 | 0 | 0 |
| 0.5 | Biphasic | 10.49 | 2.51 | 0.39 | 0.25 | 0.21 |
| 1 | Monophasic | 11.99 | 3.14 | 0.56 | 0.32 | 0.27 |
| 2 | Monophasic | 12.74 | 3.50 | 0.63 | 0.40 | 0.35 |
| 3 | Monophasic | 12.92 | 3.57 | 0.66 | 0.46 | 0.39 |
| 5 | Monophasic | 13.02 | 3.57 | 0.69 | 0.56 | 0.44 |
| 7 | Monophasic | 12.94 | 3.53 | 0.65 | 0.65 | 0.50 |
| 9 | Monophasic | 12.92 | 3.51 | 0.62 | 0.69 | 0.52 |
| 12 | Monophasic | 12.66 | 3.35 | 0.61 | 0.91 | 0.62 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 6
Influence of water

| | | Analytical data after 5 hours' reaction time | | | | | |
|---|---|---|---|---|---|---|---|
| | | | | | | | (IVa) + |
| | Proportion | | | | | | (Va) + |
| | of water at | | | | | | (VIa) + |
| | reaction | | | | | | (VIIa) + |
| Exam- | start | (Ia) | (IVa) + (Va) | (VIa) | (VIIa) | (VIIIa) | (VIIIa) |
| ple | [% by wt.] | [% by wt.] | [% by wt.] | [% by wt.] | [% by wt.] | [% by wt.] | [% by wt.] |
| 3 | 10.0 | 13.83 | 3.18 | 0.42 | 0.40 | 0.25 | 4.25 |
| 4 | 14.0 | 12.55 | 2.72 | 0.46 | 0.38 | 0.21 | 3.77 |
| 5 | 19.0 | 13.02 | 3.57 | 0.69 | 0.56 | 0.44 | 5.26 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 7a
Feedstocks (example 6)

| Starting | | | | | Mass | Molar | |
|---|---|---|---|---|---|---|---|
| weight | | | Mass | | fraction | amount | reaction |
| [g] | Feedstock | | [g] | | [% by wt.] | [mol] | temperature |
| 756.6 | Diethyl ketone (IIIa) | | 756.6 | | 65.8 | 8.78 | 90° C. |
| 117.0 | Formaldehyde | Formaldehyde (II) | 57.3 | | 5.0 | 1.91 | Molar ratio |
| | (49%) | Water | 59.7 | 165.2 | 14.4 | 9.2 | TMA[(II)] |

| | | | |
|---|---|---|---|
| 105.5 | Water | 105.5 | 1.46 |

| | | | | |
|---|---|---|---|---|
| 5.8 | Methanol | 5.8 | 0.5 | 0.18 |
| 165.1 | Trimethylamine (“TMA”) | 165.1 | 14.4 | 2.79 |

TABLE 7b
Reaction profile (example 6)

| | | | (IVa) + | | | |
|---|---|---|---|---|---|---|
| Reaction | | (Ia) | (Va) | (VIa) | (VIIa) | (VIIIa) |
| time | Phase | [% | [% | [% | [% | [% |
| [hours] | nature | by wt.] | by wt.] | by wt.] | by wt.] | by wt.] |
| 0 | Biphasic | 0 | 0 | 0 | 0 | 0 |
| 0.5 | n.d. | 8.38 | 2.20 | 0.49 | 0.16 | 0.16 |
| 1 | Monophasic | 9.59 | 2.56 | 0.60 | 0.20 | 0.18 |
| 2 | Monophasic | 10.98 | 2.94 | 0.71 | 0.26 | 0.24 |
| 3 | Monophasic | 11.65 | 3.11 | 0.76 | 0.31 | 0.27 |
| 5 | Monophasic | 11.99 | 3.22 | 0.79 | 0.37 | 0.31 |

n.d. = not determined
(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 8a
Feedstocks (example 7)

| Starting | | | | | Mass | Molar | |
|---|---|---|---|---|---|---|---|
| weight | | | Mass | | fraction | amount | reaction |
| [g] | Feedstock | | [g] | | [% by wt.] | [mol] | temperature |
| 752.8 | Diethyl ketone (IIIa) | | 752.8 | | 65.5 | 8.74 | 85° C. |
| 116.4 | Formaldehyde | Formaldehyde (II) | 57.0 | | 5.0 | 1.90 | Molar ratio |
| | (49%) | Water | 59.4 | 164.4 | 14.3 | 9.1 | TMA[(II)] |

| | | | |
|---|---|---|---|
| 105.0 | Water | 105.0 | 1.46 |

| | | | | |
|---|---|---|---|---|
| 11.5 | Methanol | 11.5 | 1.0 | 0.36 |
| 164.3 | Trimethylamine (“TMA”) | 164.3 | 14.3 | 2.78 |

TABLE 8b
Reaction profile (example 7)

| | | | (IVa) + | | | |
|---|---|---|---|---|---|---|
| Reaction | | (Ia) | (Va) | (VIa) | (VIIa) | (VIIIa) |
| time | Phase | [% | [% | [% | [% | [% |
| [hours] | nature | by wt.] | by wt.] | by wt.] | by wt.] | by wt.] |
| 0 | Biphasic | 0 | 0 | 0 | 0 | 0 |
| 0.5 | n.d. | 7.78 | 2.20 | 0.59 | 0.15 | 0.18 |
| 1 | Monophasic | 9.16 | 2.28 | 0.48 | 0.17 | 0.14 |
| 2 | Monophasic | 10.58 | 2.76 | 0.61 | 0.23 | 0.19 |
| 3 | Monophasic | 10.99 | 2.86 | 0.66 | 0.26 | 0.21 |
| 5 | Monophasic | 11.83 | 3.07 | 0.72 | 0.32 | 0.24 |

n.d. = not determined
(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 9a
Feedstocks (example 7)

| Starting | | | | | Mass | Molar | |
|---|---|---|---|---|---|---|---|
| weight | | | Mass | | fraction | amount | reaction |
| [g] | Feedstock | | [g] | | [% by wt.] | [mol] | temperature |
| 680.8 | Diethyl ketone (IIIa) | | 680.8 | | 59.2 | 7.90 | 80° C. |
| 124.4 | Formaldehyde | Formaldehyde (II) | 61.0 | | 5.3 | 2.03 | Molar ratio |
| | (49%) | Water | 63.4 | 177.1 | 15.4 | 9.8 | TMA[(II)] |

| | | | |
|---|---|---|---|
| 113.7 | Water | 113.7 | 1.35 |

| | | | | |
|---|---|---|---|---|
| 69.0 | Methanol | 69.0 | 6.0 | 2.15 |
| 162.2 | Trimethylamine (“TMA”) | 162.2 | 14.1 | 2.74 |

TABLE 9b
Reaction profile (example 8)

| | | | (IVa) + | | | |
|---|---|---|---|---|---|---|
| Reaction | | (Ia) | (Va) | (VIa) | (VIIa) | (VIIIa) |
| time | Phase | [% | [% | [% | [% | [% |
| [hours] | nature | by wt.] | by wt.] | by wt.] | by wt.] | by wt.] |
| 0 | Biphasic | 0 | 0 | 0 | 0 | 0 |
| 0.5 | Monophasic | 5.89 | 0.75 | 0.03 | 0.07 | 0.02 |
| 1 | Monophasic | 8.10 | 1.07 | 0.08 | 0.10 | 0.06 |
| 2 | Monophasic | 9.90 | 1.63 | 0.25 | 0.16 | 0.07 |
| 3 | Monophasic | 10.84 | 1.98 | 0.16 | 0.21 | 0.09 |
| 5 | Monophasic | 11.31 | 2.16 | 0.19 | 0.23 | 0.11 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 10
Influence of methanol

| | Propor- | Analytical data after 5 hours' reaction time | | | | | |
|---|---|---|---|---|---|---|---|
| | tion | | | | | | (IVa) + |
| | of | | | | | | (Va) + |
| | water at | | | | | | (VIa) + |
| | reaction | | | | | | (VIIa) + |
| | start | (Ia) | (IVa) + (Va) | (VIa) | (VIIa) | (VIIIa) | (VIIIa) |
| Example | [% by wt.] | [% by wt.] | [% by wt.] | [% by wt.] | [% by wt.] | [% by wt.] | [% by wt.] |
| 6 | 0.5 | 11.99 | 3.22 | 0.79 | 0.37 | 0.31 | 4.69 |
| 7 | 1.0 | 11.83 | 3.07 | 0.72 | 0.32 | 0.24 | 4.35 |
| 4 | 3.0 | 12.55 | 2.72 | 0.46 | 0.38 | 0.21 | 3.77 |
| 9 | 6.0 | 11.31 | 2.16 | 0.19 | 0.23 | 0.11 | 2.69 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 11a
Feedstocks (example 9)

| Starting | | | | | Mass | Molar | |
|---|---|---|---|---|---|---|---|
| weight | | | Mass | | fraction | amount | reaction |
| [g] | Feedstock | | [g] | | [% by wt.] | [mol] | temperature |
| 685.4 | Diethyl ketone (IIIa) | | 685.4 | | 59.6 | 7.96 | 70° C. |
| 117.1 | Formaldehyde | Formaldehyde (II) | 57.4 | | 5.0 | 1.91 | Molar ratio |
| | (49%) | Water | 59.7 | 211.7 | 18.4 | 11.75 | TMA[(II)] |

| | | | |
|---|---|---|---|
| 152.0 | Water | 152.0 | 1.42 |

| | | | | |
|---|---|---|---|---|
| 34.5 | Methanol | 34.5 | 3.0 | 1.08 |
| 161.0 | Trimethylamine (“TMA”) | 161.0 | 14.0 | 2.72 |

TABLE 11b
Reaction profile (example 9)

| | | | (IVa) + | | | |
|---|---|---|---|---|---|---|
| Reaction | | (Ia) | (Va) | (VIa) | (VIIa) | (VIIIa) |
| time | Phase | [% | [% | [% | [% | [% |
| [hours] | nature | by wt.] | by wt.] | by wt.] | by wt.] | by wt.] |
| 0 | Biphasic | 0 | 0 | 0 | 0 | 0 |
| 0.5 | Biphasic | 7.77 | 1.57 | 0.28 | 0.12 | 0.07 |
| 1 | Biphasic | 8.86 | 1.90 | 0.35 | 0.16 | 0.09 |
| 2 | Biphasic | 10.24 | 2.36 | 0.45 | 0.20 | 0.12 |
| 3 | Monophasic | 10.79 | 2.57 | 0.51 | 0.23 | 0.14 |
| 5 | Monophasic | 11.62 | 2.91 | 0.61 | 0.28 | 0.18 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

TABLE 12
Influence of reaction temperature

| | Analytical data after 5 hours' reaction time | | | | | | |
|---|---|---|---|---|---|---|---|
| | | | | | | | (IVa) + |
| | | | | | | | (Va) + |
| | | | | | | | (VIa) + |
| | | | | | | | (VIIa) + |
| | Reaction | | | | | | (VIIIa) |
| Exam- | temperature | (Ia) | (IVa) + (Va) | (VIa) | (VIIa) | (VIIIa) | [% |
| ple | [° C.] | [% by wt.] | [% by wt.] | [% by wt.] | [% by wt.] | [% by wt.] | by wt.] |
| 9 | 70 | 11.62 | 2.91 | 0.61 | 0.28 | 0.18 | 3.98 |
| 5 | 90 | 13.02 | 3.57 | 0.69 | 0.56 | 0.44 | 5.26 |

(Ia) 1-Hydroxy-2-methyl-3-pentanone
(IVa) 1,5-Dihydroxy-2,4-dimethyl-3-pentanone
(Va) 1-Hydroxy-2-hydroxymethyl-2-methyl-3-pentanone
(VIa) 1,5-Dihydroxy-2-hydroxymethyl-2,4-dimethyl-3-pentanone
(VIIa) 2-Methyl-1-penten-3-one
(VIIIa) 2,4-Dimethyl-5-hydroxy-1-penten-3-one

1.-15. (canceled)

16. A method for preparing a β-hydroxyketone (I) having 4 to 8 carbon atoms which comprises reacting formaldehyde (II) with a branched or unbranched dialkyl ketone (III) having 3 to 7 carbon atoms in the liquid phase in a reactor in the presence of a basic component at a temperature of 50 to 150° C. and a pressure of 0.2 to 10 MPa abs, wherein (a) a trialkylamine having 1 to 4 carbon atoms per alkyl group is used as basic component and the reaction (b) is carried out in the presence of 1 to 25% by weight water, based on the liquid phase, and (c) at a molar ratio of trialkylamine to formaldehyde (II) in the liquid phase of 1 to 5.

17. The method according to claim 16, wherein the reaction is carried out at a molar ratio of dialkyl ketone (III) to formaldehyde (II) in the liquid phase of from 1.5 to 10.

18. The method according to claim 16, wherein 1-hydroxy-2-methyl-3-pentanone (Ia) is prepared as β-hydroxyketone (I) by reacting formaldehyde (II) with diethyl ketone (IIIa) as dialkyl ketone (III).

19. The method according to claim 16, wherein the trialkylamine used is trimethylamine or triethylamine.

20. The method according to claim 16, wherein the reaction is carried out at a molar ratio of trialkylamine to formaldehyde (II) in the liquid phase of from 1 to 3.

21. The method according to claim 16, wherein the reaction is carried out in the presence of 10 to 15% by weight water, based on the liquid phase.

22. The method according to claim 16, wherein the reaction is carried out in the presence of 0.1 to 6% by weight methanol, based on the liquid phase.

23. The method according to claim 16, wherein the reaction is carried out at a temperature of 60 to 100° C.

24. The method according to claim 16, wherein the reaction is carried out at a pressure of 0.2 to 2 MPa abs.

25. The method according to claim 16, wherein the method is carried out continuously in a flow tube as reactor.

26. The method according to claim 16, wherein the method is carried out discontinuously or semi-continuously in a stirred tank as reactor.

27. The method according to claim 16, wherein the lighter boiling components than the β-hydroxyketone (I) are removed by distillation from the output of the reactor and purified β-hydroxyketone (I) is obtained as low boiler by distillation from the bottom product containing β-hydroxyketone (I).

28. The method according to claim 18 for preparing 1-hydroxy-2-methyl-3-pentanone (Ia) by reacting formaldehyde (II) with diethyl ketone (IIIa), wherein
(a) trimethylamine or triethylamine is used as trialkylamine,
(b) the reaction is carried out in the presence of 0.1 to 6% by weight methanol, based on the liquid phase,
(c) the output of the reactor is fed to a first distillation column and the trialkylamine and methanol used are removed therefrom as low boilers by distillation,
(d) the bottoms output of the first distillation column is fed to a second distillation column and diethyl ketone (IIIa) and water are removed therefrom as low boilers by distillation, and
(e) the bottoms output of the second distillation column is fed to a third distillation column and purified 1-hydroxy-2-methyl-3-pentanone (Ia) is obtained therefrom as low boiler by distillation.

29. The method according to claim 28, wherein in step (c) 90 to 100% by weight of the trialkylamine separated is fed back to the reactor.

30. The method according to claim 28, wherein the low boiler product removed in step (d) is separated into an aqueous and an organic liquid phase, comprising diethyl ketone (IIIa), and the organic liquid phase comprising diethyl ketone (IIIa) is fed back to the reactor to an extent of 90 to 100% by weight.
